(19) United States
(12) Patent Application Publication
(10) Pub. No.: US 2003/0229356 A1
(43) Pub. Date: Dec. 11, 2003

(54) Curved acetabular shell impaction instrument and method of use

(76) Inventor: Donald Dye, Pflugerville, TX (US)

Correspondence Address: Attention: Kenneth S. Barrow, Centerpulse USA Inc., Suite 1000, 12 Greenway Plaza, Houston, TX, 77046, US

(21) Appl. No.: 10/375,336

(22) Filed: Feb. 27, 2003

Related U.S. Application Data
(60) Provisional application No. 60/387,543, filed on Jun. 10, 2002.

(51) Int. Cl. A61B 17/58

(52) U.S. Cl. 606/99

(57) ABSTRACT

A method and apparatus for performing minimally invasive hip surgery for implanting a prosthetic acetabular component into a natural acetabulum. The method and apparatus include an acetabular shell impaction instrument for aligning and impacting the acetabular component into the acetabulum. The impaction instrument includes a curved section to provide access to the acetabulum through a minimally invasive incision.

### CROSS-REFERENCE TO RELATED APPLICATIONS

This application claims benefit of priority of U.S. Provisional Application Serial No. 60/387,543 filed Jun. 10, 2002.

### FIELD OF THE INVENTION

The disclosure herein generally relates to a method and apparatus for performing minimally invasive hip replacement surgery for the acetabulum using a curved acetabular shell impaction instrument for impacting an acetabular shell into the acetabulum.

### BACKGROUND OF THE INVENTION

Traditional hip replacement surgery has been used in the United States since as early as the 1960's. The surgical technique to implant a hip has not drastically changed over the years, and today, this technique is quite successful. In fact, the surgical technique is prolifically used throughout the world and has a known success rate of over 90%. Certainly, the traditional surgical technique is fundamentally sound and predictable.

Unfortunately, traditional techniques to implant a hip have well recognized shortcomings. Most importantly, a rather large incision is made on the side of the hip. The incision can extend from 6 to 12 inches; the actual length of the incision depends on the size of the patient and type of surgery (revision versus total hip arthroplasty, for example). A long, deep incision can divide a number of important stabilizing muscles and tendons and further damage the hip joint and surrounding soft tissue. Inevitably, long incisions lead to larger blood losses, longer rehabilitation times for patients, and unsightly scar lines. A patient can easily spend four or five days in the hospital after a total hip arthroplasty, for example.

Recently, surgeons have been developing new, less invasive surgical techniques to perform total hip arthroplasty and revision hip surgery. Minimally invasive surgery, or MIS, is one such technique with great promise to become a popular and accepted technique for implanting a hip.

MIS has significant advantages over traditional hip replacement surgery. Most importantly, a rather small incision is made on the side on the hip. This incision is approximately 3 to 5 inches long, and the benefits of a shorter incision are enormous.

First and foremost, the patient can recover in a much shorter period of time after a MIS. The recuperation time in the hospital can be a few days and significantly reduce the cost to both the patient and hospital. In fact, some patients are leaving the hospital within 24 to 48 hours after the surgery. Obviously, this shortened time period is extremely important to the patient.

As another advantage, MIS is less invasive and traumatic to the patient. Significantly less soft tissue is disrupted in a minimally invasive surgery compared to a traditional hip surgery. Also, the amount of blood loss is reduced, and patients will require fewer blood transfusions. Further, the length of the scar is significantly smaller, and these scars are more cosmetically appealing. The incisions themselves heal in a much shorter period of time and are much less painful than a long ten or twelve inch incision. As such, the patient can sooner return to work or enjoy recreational activities. In short, the patient can more quickly return to a normal way of life.

Presently, instruments to perform MIS are being developed and refined. These instruments have a vital role in the ability to perform a successful minimally invasive surgery. These instruments, for example, must enable the surgeon to place the hip implant in a very precise location. If the implant is not accurately placed, then complications, such as dislocation or subluxation, can occur. Further and most importantly, the instruments must consistently and reliably perform through a small three inch opening in the patient.

A successful design of instruments for MIS has other challenges as well. Specifically, the instrument must be easy to use and facilitate the implantation procedure. If the MIS instrumentation is too cumbersome or not easy to manipulate, then the surgeon will be less likely to use minimally invasive surgery. The patient, then, will not reap the benefits MIS has to offer.

As yet another consideration, MIS instrumentation must appeal to a wide range of orthopedic surgeons with various skills and experience. If, for example, the instruments are too complex and complicated, then they will not be appealing and accepted in the orthopedic surgical community. Further yet, the training and skill level required to use the instruments and become proficient with them, cannot be overly taxing on the orthopedic surgeons.

In short, instruments play a vital role in MIS surgery for hip implantation. It therefore would be advantageous to provide a new method and accompanying instruments for performing a minimally invasive surgery to implant a prosthetic hip.

### SUMMARY OF THE INVENTION

The present invention is directed to a method and apparatus for performing minimally invasive hip replacement surgery for the acetabulum using a curved acetabular shell impaction instrument for impacting an acetabular shell into the acetabulum.

The method of the present invention generally comprises the steps of templating the acetabulum to estimate the size of reamer and acetabular components; incising the surgical site with a single incision approximately three inches in length; exposing the acetabular joint and dislocating the hip from the acetabulum; providing an acetabular reamer; reaming the acetabulum with the reamer; providing an acetabular shell impaction instrument; inserting and aligning a trial shell into the reamed acetabulum; inserting a trial insert to the trial shell; removing the trial insert and shell; inserting and aligning an implant shell into the reamed acetabulum; impacting the implant shell with the acetabular shell impaction instrument; inserting and impacting an implant insert into the implant shell; and closing the surgical site.

One important advantage of the present invention is that the method and acetabular shell impaction instrument are used in a minimally invasive orthopedic hip surgery. A single, small three inch incision is made at the surgical site on the side on the hip. The method of the present invention, thus, enjoys the benefits of a shorter incision compared to traditional hip surgery that uses a much longer incision. As one benefit, the patient can recover in a much shorter period of time after a MIS. The recuperation time in the hospital can be a few days and significantly reduce the cost to both the patient and hospital. This shortened time period is extremely important to the patient. Further, MIS is less invasive and traumatic to the patient. Significantly less soft tissue is disrupted in a minimally invasive surgery compared to a traditional hip surgery. Also, the amount of blood loss is reduced, and patients will require fewer blood transfusions. Further, the length of the scar is significantly smaller, and these scars are more cosmetically appealing. The incisions themselves heal in a much shorter period of time and are much less painful than a long ten or twelve inch incision. As such, the patient can sooner return to work or enjoy recreational activities. In short, the patient can more quickly return to a normal way of life.

Another important advantage of the present invention is that a curved acetabular shell impaction instrument is used. The curvature of this instrument is specifically designed and adapted to be used in minimally invasive surgical techniques for impacting an acetabular shell into the natural acetabulum of a patient. While connected to an acetabular shell positioned in the acetabulum, the instrument is shaped to avoid the edges of the incision or wound.

The acetabular shell impaction instrument generally comprises an elongated body that extends from a proximal impaction end to a distal connection end. The impaction end includes an enlarged head adapted to receive the impact of a hammer or mallet. A tapering section connects to the center of the head and extends outwardly therefrom and transitions into a curved section having a circular or elliptical shape. The connection end includes a bore that extends from the tip of the end to a channel on the underside of the body. The bore is sized and shaped to house a bolt or screw that is captured inside the bore and floats or moves therein to threadably engage and attach to an acetabular shell. A guiding rod may be removeably attached to the body of the instrument. The rod is adapted to help move and align the acetabular shell while it is being positioned into the natural acetabulum.

The acetabular shell impaction instrument of the present invention enables the surgeon to impact precisely the implanted shell at the correct angular orientation. Force is distributed from the proximal impaction end to the center of the shell. Further, the shell can be accurately placed since the instrument is adapted to move and align the shell while it is being positioned in the acetabulum. As such, the likelihood of a complication associated with an incorrectly aligned acetabular implant is reduced.

As another advantage, the acetabular shell impaction instrument can consistently and reliably perform through a small three inch opening in the patient. The instrument is formed of a strong cast frame through which the impact of a hammer can be transmitted to the acetabular shell.

Further yet, the instrument is easy to use and facilitates the implantation procedure. As such, the acetabular shell impaction instrument can appeal to a wide range of orthopedic surgeons with various skills and experience. Further yet, the training and skill level required to use the instrument and become proficient with it is not overly taxing on the orthopedic surgeon.

### BRIEF DESCRIPTION OF THE DRAWINGS

FIG. 1 is a view of a patient showing a femur and femoral head positioned in the acetabulum with an MIS incision marked along the hip.

FIG. 2 is a view of an acetabular shell embedded in the acetabulum with an acetabular insert being connected to the shell.

FIG. 3 is a side perspective view of the curved shell impaction instrument of the present invention implanting an acetabular shell into the acetabulum.

FIG. 4 is a top view of the curved shell impaction instrument.

FIG. 5 is a front view of the curved shell impaction instrument.

### DETAILED DESCRIPTION

The instruments, method, and steps of the present invention are now described in more detail. The method describes the steps to perform a minimally invasive surgery to implant a prosthetic acetabular component into the natural acetabulum of a patient. Some of these steps described in the method are known to those skilled in the art and will not be discussed in great detail. Further, one skilled in the art will appreciate that certain steps may be altered or omitted while other steps may be added without departing from the scope of the invention. The novel steps of the present invention, for example, can be applied to total hip arthroplasty, to revision surgeries for total and partial hip replacement, and to other orthopedic surgeries using minimally invasive surgical techniques.

To facilitate a discussion of the present invention, the method of implanting a prosthetic acetabular component is divided into a plurality of steps or sections. Each of these sections is discussed seriatim.

More specifically, the method of the present invention teaches how to implant a prosthetic acetabular shell and insert into the natural acetabulum using a curved shell impaction instrument. For illustrative purposes, the discussion focuses on implanting a Converge™ Acetabular System of Centerpulse Orthopedics Inc. of Austin, Tex. This system illustrates one possible acetabular system that can be used. One skilled in the art will appreciate that other, different acetabular systems can also be used with the method and apparatus of the present invention without departing from the scope of the invention.

Templating the Acetabulum

Typically, the side of the acetabulum to be reconstructed is templated. Use of a template enables the surgeon to make an estimation of the size of reamers to be used and the size of acetabular component to be inserted. The acetabulum is templated on the both the anterior-posterior (A/P) and lateral radiographs. The hemisphere of the acetabular component is aligned with the mouth of the bony, natural acetabulum while simultaneously avoiding any osteophytes. On the A/P radiograph, the acetabular component should rest on the floor of the cotyloid notch and may touch the illoischial line. Further, the component should have a maximum lateral opening of about 40°. On the groin lateral radiograph, the cup size selected should contact the anterior and posterior rim of the bony, natural acetabulum and the medial subchondral bone. A correct position of the acetabular component will anatomically reproduce the center of rotation of the femoral head. If a bony defect is identified, use the correctly placed template to measure for proper size of the acetabular component and determine any need for bone graft.

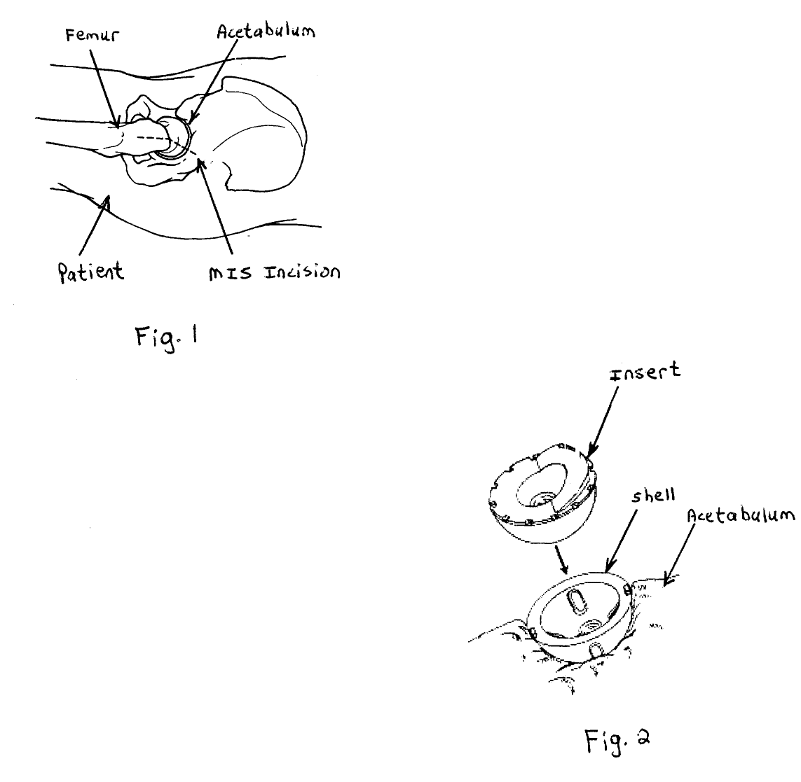

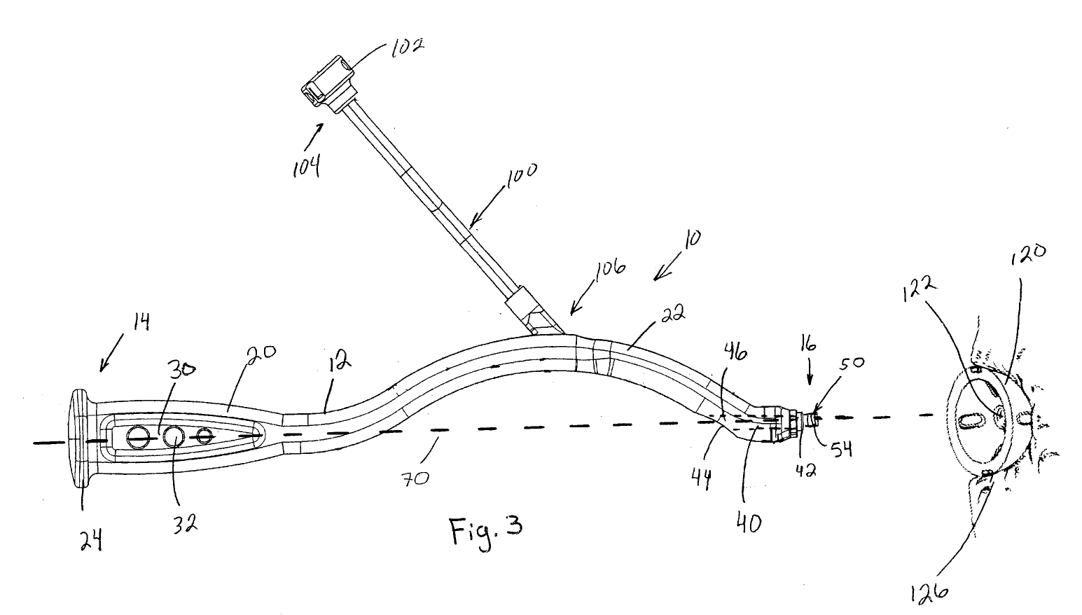

Incising the Surgical Site (See FIG. 1)

A relatively small, single minimally invasive incision is made at the surgical site. A minimally invasive incision for this procedure has a length from about 2½ inches to about 4 or 5 inches. The incision is slightly curved or straight, commences near the vastus tubercle, and continues toward the greater trochanter and posterior inferior spine. The incision should be carried down through subcutaneous tissue and fascia lata. Any muscle tissue should be gently split in line with its fibers. At this time, a leg length measurement can be taken using techniques known in the art.

Providing Retractors

The retractors have an elongated, flat, thin body with two primary sections, a handle section and a retracting section. The handle section is elongated and adapted to be gripped with a hand. A smooth curved section transitions the handle section to the retracting section. The retracting section has a paddle with a prong that curves outwardly and away from the paddle and handle section.

Exposing the Acetabular Joint & Dislocating the Hip from the Acetabulum

Next, the knee is flexed, and the leg is internally rotated. Using a hot knife, the piriformis, short external rotators, quadratus femoris, and some posterior capsule are incised off the posterior trochanter to expose the lesser trochanter. Dislocation of the hip can now occur. A bone hook or skid may be used to avoid excess torsion on the femoral shaft.

At this time, retractors may be placed, for example under the femoral head or lesser trochanter, in order to achieve visualization for proper transection of the femoral neck if this procedure is desired at this time. If such transection occurs, the femoral neck should be transected at the templated level. Then retract the femur in an anterior direction to expose the acetabulum. Care should be taken to protect the sciatic nerve.

A retractor can be placed on the pelvis to hold the femur in an anterior position to the acetabulum. The capsule can be retracted in the posterior using retractors or pins. After the labrum and osteophytes are removed, at least a partial view of the acetabulum should be available.

Providing an Acetabular Reamer

An acetabular reamer is provided to ream the natural acetabulum. The reamer is designed and adapted to be used with minimally invasive surgical techniques of the acetabulum. Specifically, the reamer is shaped to fit through the small incision at the surgical site. Further, the reamer is angled so the distal end properly engages the natural acetabulum with the correct angular orientation and without disrupting the incision and surrounding soft tissue.

Reaming the Acetabulum

Reaming of the acetabulum should begin with a reamer that is two sizes smaller than the preoperatively selected acetabular component size. A smaller reamer ensures that the fit does not exceed the anterior-posterior diameter. Of course, the reamer should not be so small that excessive anterior or posterior reaming occurs.

After an appropriately sized reamer is connected to the acetabular reamer, reaming should begin transversely toward the cotyloid notch. The ridges of the horseshoe (or medial osteophytes) should be removed. Reaming then continues in the position of desired anteversion while simultaneously creating a hemisphere. Larger reamers are used until the anterior and posterior rim of the acetabulum is contacted. The reamer should not be sunk below the superior rim of the bony acetabulum or reamed through the cortical bone of the cotyloid notch. Cancellous bone will be evident where the horseshoe ridges have been removed. The proper size trial shell should be selected according to the size of the reamer.

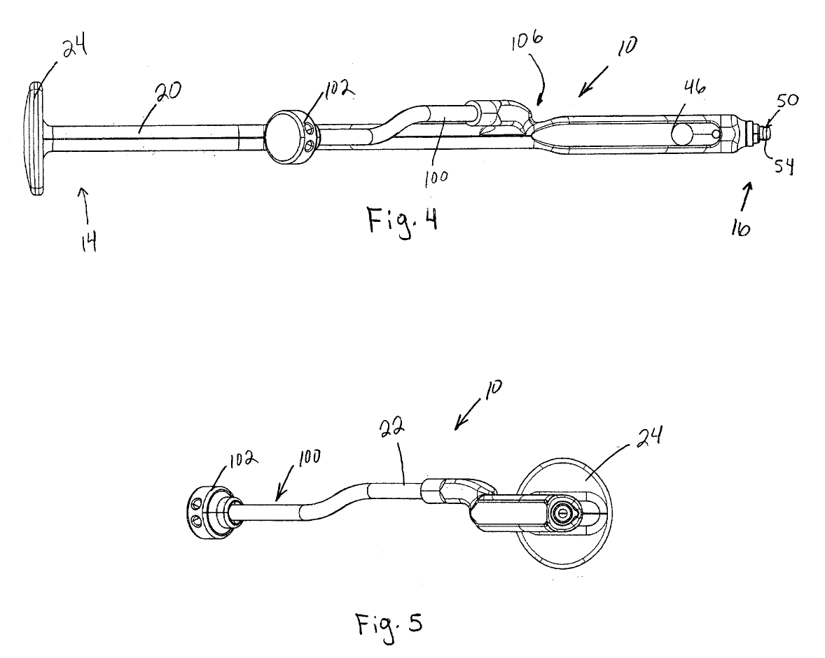

Providing an Acetabular Shell Impaction Instrument (See FIGS. 3-5)

An acetabular shell impaction instrument is provided to align and then impact the acetabular shell into the natural acetabulum. The instrument is designed and adapted to be used with minimally invasive surgical techniques of the acetabulum. Specifically, the instrument has a curved shape to fit through the small incision at the surgical site and precisely impact the acetabular shell at the correct angular orientation. Further, this curvature enables the instrument to engage the shell in the acetabulum without disrupting the edges of the incision and surrounding soft tissue. Further yet, the instrument is adapted to move and align the acetabular shell while it is positioned in the acetabulum. It is important to position properly the shell before it is impacted and permanently seated in the acetabulum. The acetabular shell impaction instrument of the present invention is discussed in more detail with reference to FIGS. 3-5.

Inserting a Trial Shell into the Acetabulum

The acetabular shell impaction instrument keys off the dome of the trial shell and is threaded or engaged in place. The instrument may offer anteversion and abduction references and rotational control. Preferably, the distal end of the instrument is adapted to mate with both the trial shell and implant shell in one single orientation. To connect the components, the distal end of the instrument is keyed and threadably attached to the trial shell. One skilled in the art will appreciate that the instrument, inserts, and shells can connect in various ways.

After the trial shell is inserted into the acetabulum, its position is verified through a trial window. The edge of the trial shell should be level with the anterior-inferior margins of the acetabulum and should completely fill the anterior-posterior bony acetabulum. The instrument can be used to move and align shell while it is positioned in the acetabulum. At this time, the trial shell can be manually tested to ensure that it is stable. If the trial is loose, then use the next larger size. If the trial is too tight, then ream the rim of the acetabulum. Importantly, the trial shell should be stable before selecting a similarly sized acetabular implant shell.

Inserting a Trial Inserting into the Trial Shell

Now, the trial insert is ready to be placed in the trial shell. An instrument is engaged in the rim of the trial insert and it is positioned inside the cavity of the trial shell. The trial insert contains a captured screw at the apex and can be threaded into the dome of the trial shell with a screwdriver or other tool. The trial components should be checked for proper fit and size.

At this point, the trials are removed from the surgical site. One skilled in the art, though, will appreciate that the trials could be temporarily left inserted to the natural acetabulum to articulate with a trial femoral prosthesis in a total hip replacement surgery.

Inserting an Implant Shell into the Acetabulum (See FIG. 3)

Some implant shells may be provided with flared rims and outer bone engaging spikes. In order to insert such a shell, cancellous bone slurry may be added within the acetabulum to fill existing bone cysts and provide an interface layer. Addition of this slurry typically occurs in total hip arthroplasty situations.

The acetabular implant shell is positioned into the acetabulum using the same acetabular shell impaction instrument used with the trial shell. Specifically, the distal connection end of the instrument is engaged and connected to the shell. The shell is partially inserted into the acetabulum until the rim begins to engage bone. The implant is then positioned with the instrument to the desired angular orientation, such as abduction and anteversion. Preferably, the shell is positioned with 20° to 25° of anteversion and with an abduction angle of about 35° to 45°. The anteversion can be verified using techniques known to those skilled in the art. The proximal impaction end of the instrument is then impacted with a mallet or similar instrument. Force from the mallet is transferred from the instrument to the shell as it is driven and permanently seated into the natural acetabulum. The shell should be driven into the acetabulum until the outer fixation spikes centrally engage into cancellous bone.

Removing Screw-Holes & Inserting Dome Plug

The implant shell may be provided with screw-hole seals and a dome plug. In this instance, after the shell is properly seated in the acetabulum, one or more of the screw-hole seals may be removed with a screw-hole extracting instrument. This instrument is inserted through the incision and into the indentation of the screw-hole seal. Leverage is used to -dislodge the screw-hole seal from the shell. It should be noted that screw-hole seals can be dislodged at the back table before the shell is seated in the acetabulum. By contrast, the dome plug should be installed before the insert is impacted.

Drilling Holes & Attaching Bone Screws

Next, a drill bit is connected to a flexible driver and is positioned into the selected screw hole at an angle up to about 16°. As the hole is drilled, care should be taken to protect the sciatic nerve and superior gluteal artery. A depth gauge may be inserted into the drilled holes to determine the depth for a corresponding bone screw. If desired, a tapping bit may be connected to the driver to tap the hole.

A bone screw is connected to a U-joint screwdriver and positioned into the drilled hole. The screw should be seated into the countersunk holes of the shell so the acetabular insert can properly snap into the shell.

Inserting & Impacting Insert into Shell

Various inserts known to those skilled in the art (such as standard, hooded, and protrusion inserts) can be inserted into the implant shell. Once the appropriate size and style insert is selected, the insert is connected to an instrument. The insert is positioned into the cavity of the shell and should be rotated to align with the antirotational pegs on the shell. A surgical mallet is used to strike the proximal end of the instrument to seat the insert into the shell.

Closing Surgical Site

Once the insert is firmly connected to the shell, all instruments and devices are removed from the site. The acetabular shell and insert should now be properly positioned. Closure of the site may occur with well known techniques, such as posterior and anterior lateral approaches. Further, this disclosure will not discuss post-operative protocol or rehabilitation as such procedures are known in the art and tailored to meet the specific needs of the patient.

Detailed Description of Acetabular Shell Impaction Instrument

One important advantage of the present invention is that a curved acetabular shell impaction instrument is used. This instrument is specifically designed and adapted to be used in minimally invasive surgical techniques for aligning and impacting a prosthetic acetabular shell into the natural acetabulum of a patient.

FIGS. 3-5 show the acetabular shell impaction instrument 10 of the present invention. Instrument 10 has an elongated body 12 that extends from a proximal impaction end 14 to a distal connection end 16. The body has two primary and different sections, a straight section 20 and a curved section 22. The impaction end 14 has an enlarged round head 24 that connects to the straight section 20 of the body. Head 24 is adapted to receive striking impacts from a surgical mallet or hammer.

The straight section 20 tapers or narrows as it extends outwardly from the impaction end and toward the connection end. An elongated, frusto-conical recess 30 is formed in the straight section. This recess 30 may includes a plurality of bores 32 extending through the body.

The connection end 16 includes a bore 40 that extends completely through the body 12. Bore 40 extends from a first end 42 at the distal tip of the connection end and terminates at a second end 44 in the curved section 22 of the body. This second end forms an channel 46 in the body. Channel 46 commences with a gradual taper and deepens to form a cylindrical shape with smooth walls. The channel makes a smooth transition into bore 40.

A bolt or screw 50 is captured inside of bore 40 and cannot fall out or dislodge from the bore. The bolt has a head (not shown) and an externally threaded shaft 54 at a distal end. The head includes an internal channel or recess that is adapted to receive the driving end of an instrument. The bolt is sized and shaped to float or slideably move inside the bore. Preferably, bolt 50 should be permanently affixed in the bore and not removeable therefrom. As shown, threaded shaft 54 extends outwardly from the first end 42 of the bore. The head of the bolt extends into channel 46 and is accessible from this channel.

The curved section 22 is sized and shaped so the connection end 16 can properly align into the natural acetabulum while the impaction end 14 is outside of the wound or incision. Further, this size and shape enables the shell impaction instrument to transport, align, and drive a shell into the natural acetabulum without disrupting or affecting the edges of the incision or other soft tissue. In order to achieve this function, the shell impaction instrument has a curved shape along the curved section 22. The curved section has an arc, elliptical, or circular shape with a constant curvature. Preferably, the entire instrument is about 14 inches; and the curved section has a length of 7 inches, with the length generally between about 6 inches and 8 inches. Additional, the curved section has a curvature of 8 inches with a range between about 6 inches to 10 inches. Generally, the curved section is about one-half of the entire length of the body 12.

One skilled in the art will appreciate that the curved section can have other embodiments as well. For example, the curved section can have a substantially elliptical, arc, or circular shape. Additionally, these curvatures could be distorted, elongated, twisted, offset, or the like. Furthermore, the curved section could have other curved shapes and still be within the scope of the invention. An “S” shape is one example.

Looking now to the shell impaction instrument as a whole, the body 12 has an elongated shape with a longitudinal or central axis 70 (FIG. 3). This axis centrally extends through the impaction end 14, along the straight section 20, and out through the connection end 16. As shown then, the impaction end and the connection end are aligned and positioned along the longitudinal axis. When an impaction force is applied to the impaction end 14, the force travels along the straight section 20, up and around through the curved section 22, and out through the connection end 16. It also should be noted that a longitudinal or central axis drawn through the connection end is generally perpendicular to a plane passing through the head 24 of the impaction end. As such, force impacted on the head is transmitted centrally through the connection end and centrally into the shell even though the curved section 20 is substantially outside of the longitudinal axis 70.

General alignment of the impaction and connection ends helps to ensure that the impaction force properly transfers to the connection end so the shell can be easily and correctly driven into the acetabulum. In the preferred embodiment, then, the ends are aligned, but the invention could include offset ends. For example, the longitudinal axis could extend through the impaction end and straight section and not extend through the connection end. In this instance, the connection end would be offset from the impaction end. The connection end, for instance, could have an offset of about 5°.

Preferably, the body 12 is constructed of a single, cast steel frame through which impaction forces transmit to the shell. This construction enables complex shapes to be easily and economically produced. Further, this construction effectively transmits an audible change in pitch when the shell becomes fully impacted into the prepared acetabulum. This change in pitch is particularly advantageous in a minimally invasive surgery since the surgeon will not be able to fully see the shell in the acetabulum through the small incision. As such, the surgeon can hear when the shell is fully seated in the acetabulum because the shell impaction instrument will change its pitch as it is being struck.

The shell impaction instrument can also include an alignment or guide rod 100. The rod has an elongated straight body with a handle 102 at a proximal end 104. A distal end 106 connects to the curved section 22. The guide may be removeably connected or permanently connected to the shell impaction instrument.

The guide functions to guide placement of the connection 16 during use. As such, the surgeon can more easily ensure that the shell will be properly placed into the natural acetabulum as the shell is positioned through the small incision. The guide, for example, can assist in rotating, moving, and aligning the shell while it is in the surgical site.

In use, the shell impaction instrument is removeably connectable to the shell. As shown in FIG. 3, shell 120 includes a threaded bore 122 in the top of the dome. Bolt 50 is threadably engaged with bore 122 so the impaction instrument can hold, carry, and align the shell into the acetabulum 126. Once the shell is in position, the impaction end 14 is struck and the shell is impacted into the acetabulum. The bolt 50 is then threadably disengaged from the shell, and the instrument is removed.

It should be emphasized that although the method of the present invention was described with a specific number and sequence of steps, these steps can be altered or omitted while other steps may be added without departing from the scope of the invention. As such, the specific steps discussed in the preferred embodiment of the present invention illustrate just one example of how to utilize the novel method and steps of the present invention. Further, although illustrative embodiments and methods have been shown and described, a wide range of modifications, changes, and substitutions is contemplated in the foregoing disclosure and in some instances, some features of the embodiments or steps of the method may be employed without a corresponding use of other features or steps. Accordingly, it is appropriate that the appended claims be construed broadly and in a manner consistent with the scope of the embodiments disclosed herein.

What is claimed is:

1. A method for using minimally invasive surgery to implant a prosthetic acetabular shell and insert into a natural acetabulum, comprising the steps of:
incising a hip with a minimally invasive incision;
exposing at least a portion of an acetabular joint through the incision;
providing an acetabular impaction instrument having an elongated body extending from an impaction end to a connection end, the body having an enlarged arc connected to the connection end, wherein the arc is adapted to at least partially fit into the incision;
attaching the acetabular shell to the connection end of the acetabular impaction instrument;
positioning the acetabular shell into the natural acetabulum with the acetabular impaction instrument;
impacting the impaction end of the acetabular impaction instrument to drive the acetabular shell into the natural acetabulum;
disengaging the acetabular impaction instrument from the acetabular shell while the acetabular shell is seated in the natural acetabulum;
removing the acetabular impaction instrument form the incision; and
closing the incision.

2. The method of claim 1 further comprising the step of providing the body of the acetabular impaction instrument with two sections that form substantially an entire length of the body with the arc forming one of the sections.

3. The method of claim 1 further comprising the step of providing the body of the acetabular impaction instrument with a longitudinal axis that extends through the impaction and connection ends.

4. The method of claim 3 further comprising the step of providing the arc with a curved section that substantially falls outside of the longitudinal axis.

5. The method of claim 1 further comprising the step of providing the arc to form approximately one half of a length of the body.

6. The method of claim 1 wherein the step of incising a hip creates the minimally invasive incision with a length of about 2½ inches to about 4 to 5 inches.

7. The method of claim 1 further comprising the step of positioning at least a portion of the arc into the minimally invasive incision in order to perform the step of positioning the acetabular shell into the natural acetabulum.

8. A method for implanting a prosthetic acetabular component into a natural acetabulum of a patient, the method comprising the steps of:
incising a hip to provide surgical access to an acetabular joint;
providing an acetabular impaction instrument having a body extending from an impaction end to a connection end, the body having a curved section connected to the connection end and a straight section connected to the impaction end;
attaching the acetabular component to the connection end of the acetabular impaction instrument;
positioning the acetabular component into the natural acetabulum with the acetabular impaction instrument until at least a portion of the curved section is positioned into the patient;
impacting the impaction end of the acetabular impaction instrument to drive the acetabular component into the natural acetabulum;
disengaging the acetabular impaction instrument from the acetabular component while the acetabular component is embedded in the natural acetabulum;
removing the acetabular impaction instrument from the patient; and
closing the incision.

9. The method of claim 8 further comprising the step of providing the curved section to have substantially a shape of a partial ellipse.

10. The method of claim 8 wherein the step of incising a hip produces an incision with a length of about 2½ inches to about 5 inches.

11. The method of claim 10 further comprising the step of providing the curved section to have a shape adapted to position the acetabular component into the natural acetabulum while the straight section is positioned outside of the patient.

12. The method of claim 8 further comprising the step of providing the curved section to have a length of about 6 inches to 8 inches.

13. The method of claim 12 further comprising the step of providing the curved section to have a curvature of about 6 inches to 10 inches.

14. The method of claim 13 further comprising the step of aligning the acetabular component in the natural acetabulum with the acetabular impaction instrument before the step of impacting the impaction end of the acetabular impaction instrument.

15. A method for implanting a prosthetic acetabular component into a natural acetabulum of a patient, the method comprising the steps of:
incising the patient with an incision from about 2½ inches to about 5 inches to provide surgical access to an acetabular joint;
preparing the natural acetabulum to receive the acetabular component;
providing an acetabular impaction instrument having a body extending from an impaction end to a connection end, the body having a curved section adjacent the connection end;
attaching the acetabular component to the connection end of the acetabular impaction instrument;
positioning the acetabular component into the natural acetabulum with the acetabular impaction instrument until at least a portion of the curved section is positioned into the incision;
impacting the impaction end of the acetabular impaction instrument to drive the acetabular component into the natural acetabulum;
disengaging the acetabular impaction instrument from the acetabular component while the acetabular component is embedded in the natural acetabulum;
removing the acetabular impaction instrument from the patient; and
closing the incision.

16. The method of claim 15 further comprising the step of providing the curved section to have a length of about 7 inches.

17. The method of claim 16 further comprising the step of providing the curved section to have a curvature of about 8 inches.

18. The method of claim 17 further comprising the step of providing the curved section with a substantially constant curvature.

19. The method of claim 17 further comprising the step of providing the body with a longitudinal axis extending through both the impaction and connection ends.

20. The method of claim 15 further comprising the step of providing the curved section to have a shape selected from one of an ellipse, arc, and partial circle.
